(19) United States
(12) Patent Application Publication
(10) Pub. No.: US 2023/0158183 A1
(43) Pub. Date: May 25, 2023

(54) STETHOSCOPE STERILIZATION DEVICE AND A METHOD THEREOF

(71) Applicant: Rajeev C. Nagarad, Montgomery, AL (US)

(72) Inventor: Rajeev C. Nagarad, Montgomery, AL (US)

(21) Appl. No.: 17/530,493

(22) Filed: Nov. 19, 2021

(51) Int. Cl. A61L 2/04; A61L 2/18

(52) U.S. Cl. CPC A61L 2/04; A61L 2/18; A61L 2202/24; A61L 2202/14

(57) ABSTRACT

A stethoscope sterilization device is disclosed. A base unit adapted to accommodate a stethoscope. A diaphragm attachment assembled with the base unit, wherein the diaphragm attachment comprises an aperture, wherein the diaphragm attachment provides a surface for placing a diaphragm of the stethoscope. A heating element provided to heat the surface of the diaphragm to maintain a desirable temperature. A luminometer to monitor the diaphragm of the stethoscope for determining contamination in real-time. A dispensing unit positioned underneath the diaphragm attachment, wherein the dispensing unit is configured to dispense a disinfectant solution stored in a disinfectant reservoir over the surface of the diaphragm of the stethoscope for the sterilization via the aperture. A sponge placed over the base unit, wherein the sponge enables wiping off of excess disinfectant solution accumulated over the surface of the diaphragm of the stethoscope upon sterilization.

### BACKGROUND

Embodiments of the present disclosure relate to a medical apparatus and more particularly to a stethoscope sterilization device and a method thereof.

Stethoscope is the most used instrument in any medical environment from private clinics to general wards in hospitals. The stethoscope is a device for amplifying and transmitting various bodily sounds such as those of the heart, lungs, intestines, and blood flow in arteries and veins. The cleanliness of the stethoscope is of great concern, especially in hospitals, where infectious agents can pass easily from one patient to the next through contact with contaminants on a doctor's stethoscope. Generally, head of the stethoscope is particularly prone to contamination since it comes into physical contact with each patient. As a result, various devices are developed for performing stethoscope disinfection, to prevent unnecessary spreading of infections.

Conventionally, the device available for stethoscope disinfection includes an ultraviolet (UV) radiation-based disinfection device. However, such a conventional UV radiation-based disinfection device emits harmful radiation to disinfect the stethoscope which is harmful for living organism. Also, such a conventional device is generally designed to be of a “table-top” type construction and is not adapted to be worn by the user since they require bulky power supply. Moreover, such a conventional device has a complicated design and is much more expensive for disinfecting the stethoscope.

Hence, there is a need for an improved stethoscope sterilization device and a method thereof in order to address the aforementioned issues.

### BRIEF DESCRIPTION

In accordance with an embodiment of the present disclosure, a stethoscope sterilization device is disclosed. The device includes a base unit adapted to accommodate a stethoscope, wherein the base unit comprises at least two protrusions to grab the stethoscope within a predefined position. The device also includes a diaphragm attachment assembled with the base unit, wherein the diaphragm attachment comprises an aperture, wherein the diaphragm attachment provides a surface for placing a diaphragm of the stethoscope. The device also includes a heating element operatively coupled to the diaphragm attachment, wherein the heating element is provided to heat the surface of the diaphragm to maintain a desirable temperature. The device also includes a luminometer operatively coupled to the diaphragm attachment, wherein the luminometer is configured to monitor the diaphragm of the stethoscope for determining contamination in real-time. The device also includes a dispensing unit positioned underneath the diaphragm attachment, wherein the dispensing unit is configured to dispense a disinfectant solution stored in a disinfectant reservoir over the surface of the diaphragm of the stethoscope for the sterilization via the aperture. The device also includes a sponge placed over the base unit, wherein the sponge enables wiping off of excess disinfectant solution accumulated over the surface of the diaphragm of the stethoscope upon sterilization.

In accordance with another embodiment of the present disclosure, a method for constructing a stethoscope sterilization device is disclosed. The method includes accommodating a stethoscope by a base unit, wherein the base unit comprises at least two protrusions to grab the stethoscope within a predefined position. The method also includes assembling a diaphragm attachment with the base unit, wherein the diaphragm attachment comprises an aperture, wherein the diaphragm attachment provides a surface for placing a diaphragm of the stethoscope. The method also includes providing a heating element coupled with the diaphragm attachment to heat the surface of the diaphragm to maintain a desirable temperature. The method also includes monitoring, by a luminometer, the diaphragm of the stethoscope for determining contamination in real-time. The method also includes dispensing, by a dispensing unit positioned underneath the diaphragm attachment, a disinfectant solution stored in a disinfectant reservoir over the surface of the diaphragm of the stethoscope for the sterilization via the aperture. The method also includes enabling, by a sponge placed over the base unit, wiping off of excess disinfectant solution accumulated over the surface of the diaphragm of the stethoscope upon sterilization.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation of a stethoscope sterilization device in accordance with an embodiment of the present disclosure;

FIG. 2 represents a schematic representation of an embodiment of a stethoscope sterilization device in accordance with an embodiment of the present disclosure;

FIG. 3 is a flow chart representing the steps involved in a method for constructing a stethoscope sterilization device in accordance with the embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a stethoscope sterilization device. The device includes a base unit adapted to accommodate a stethoscope, wherein the base unit comprises at least two protrusions to grab the stethoscope within a predefined position. The device also includes a diaphragm attachment assembled with the base unit, wherein the diaphragm attachment comprises an aperture, wherein the diaphragm attachment provides a surface for placing a diaphragm of the stethoscope. The device also includes a heating element operatively coupled to the diaphragm attachment, wherein the heating element is provided to heat the surface of the diaphragm to maintain a desirable temperature. The device also includes a luminometer operatively coupled to the diaphragm attachment, wherein the luminometer is configured to monitor the diaphragm of the stethoscope for determining contamination in real-time. The device also includes a dispensing unit positioned underneath the diaphragm attachment, wherein the dispensing unit is configured to dispense a disinfectant solution stored in a disinfectant reservoir over the surface of the diaphragm of the stethoscope for the sterilization via the aperture. The device also includes a sponge placed over the base unit, wherein the sponge enables wiping off of excess disinfectant solution accumulated over the surface of the diaphragm of the stethoscope upon sterilization.

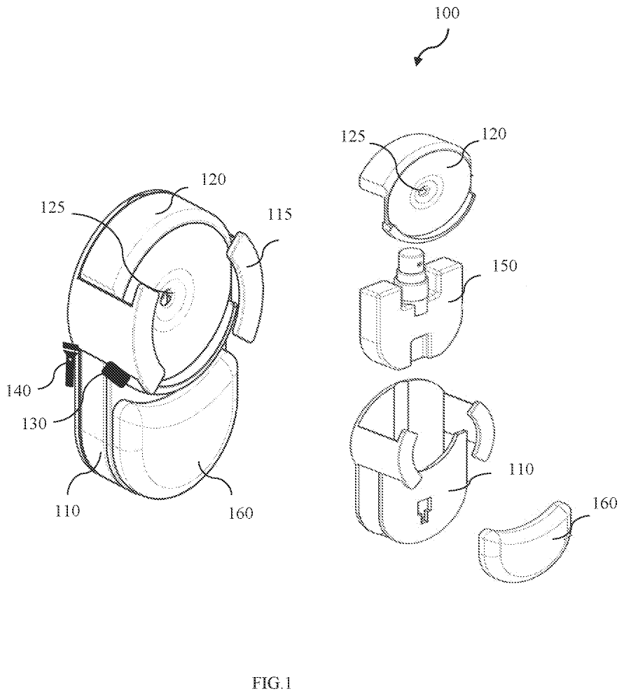

FIG. 1 is a schematic representation of a stethoscope sterilization device 100 in accordance with an embodiment of the present disclosure. The device 100 includes a base unit 110 adapted to accommodate a stethoscope, wherein the base unit comprises at least two protrusions 115 to grab the stethoscope within a predefined position. In one embodiment, the at least two protrusions 115 may include at least two C-shaped protrusions to snap up the stethoscope. In such embodiment, the at least two protrusions grab the stethoscope by enabling pushing and downward movement of the diaphragm of the stethoscope via a spring mechanism. In some embodiment, the spring mechanism enables conduction of the diaphragm of the stethoscope from an upward position to downward position thereby triggers the dispensing unit.

The device 100 also includes a diaphragm attachment 120 assembled with the base unit 110 wherein the diaphragm attachment 120 comprises an aperture 125, wherein the diaphragm attachment 120 provides a surface for placing a diaphragm of the stethoscope.

The device 100 also includes a heating element 130 operatively coupled to the diaphragm attachment. The heating element 130 is provided to heat the surface of the diaphragm to maintain a desirable temperature. In one embodiment, the heating element includes a coil of heat conducting and transferring heat through a wire wrapped around the diaphragm of the stethoscope. The device 100 also includes a luminometer 140 operatively coupled to the diaphragm attachment, wherein the luminometer is configured to monitor the diaphragm of the stethoscope for determining contamination in real-time. In a specific embodiment, the luminometer may include an adenosine triphosphate (ATP) luminometer.

The device 100 also includes a dispensing unit 150 positioned underneath the diaphragm attachment 120. The dispensing unit 150 is configured to dispense a disinfectant solution stored in a disinfectant reservoir over the surface of the diaphragm of the stethoscope for the sterilization via the aperture 125. The dispensing unit is adapted to compress when the diaphragm of the stethoscope is conducted from the upward position to the downward position. The dispensing unit 150 is also adapted to enable collection of the disinfectant solution from the disinfectant reservoir via an inlet pipe. Further, the dispensing unit 150 dispenses the disinfectant solution collected over the surface of the diaphragm of the stethoscope via the aperture in a predefined direction to sterilize the diaphragm of the stethoscope.

The device 100 also includes a sponge 160 placed over the base unit 110, wherein the sponge 160 enables wiping off of excess disinfectant solution accumulated over the surface of the diaphragm of the stethoscope upon sterilization. In one embodiment, the disinfectant solution may include isopropyl alcohol solution. In a particular embodiment, the base unit 110, the diaphragm attachment 120 and the dispensing unit 150 is fabricated from plastic.

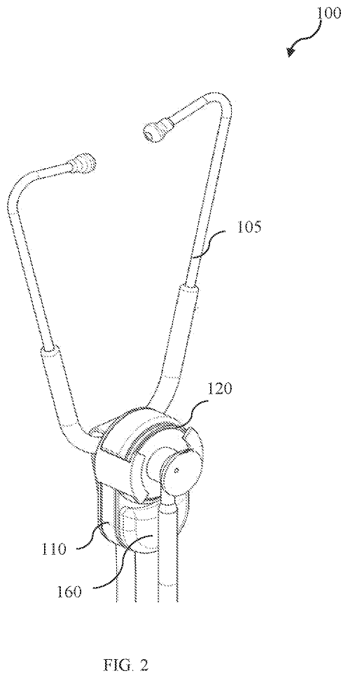

FIG. 2 represents a schematic representation of an embodiment of a stethoscope sterilization device with a stethoscope in accordance with an embodiment of the present disclosure. The device 100 for sterilization of the stethoscope 105 has 5 major components such as a base unit 110, a diaphragm attachment 120, a heating element 130, a luminometer 140, a dispensing unit or a pump 150 and a sponge 160. The device 100 attaches to the diaphragm of the stethoscope 105 and sterilizes it prior to use on a new patient decreasing spread of infection. It can also be used as a stethoscope holder of sterilization. The device 100 attaches to the stethoscope and the diaphragm sits in the base when on in use. When the stethoscope is being removed for use the dispensing unit 150 or the pump dispenses a small amount of disinfecting solution such as isopropyl alcohol on to the surface of the diaphragm. The excess solution can be wiped off with the sponge provided. The pump can store enough solution for up to 60 uses and can be easily refilled with readily available disinfecting solution.

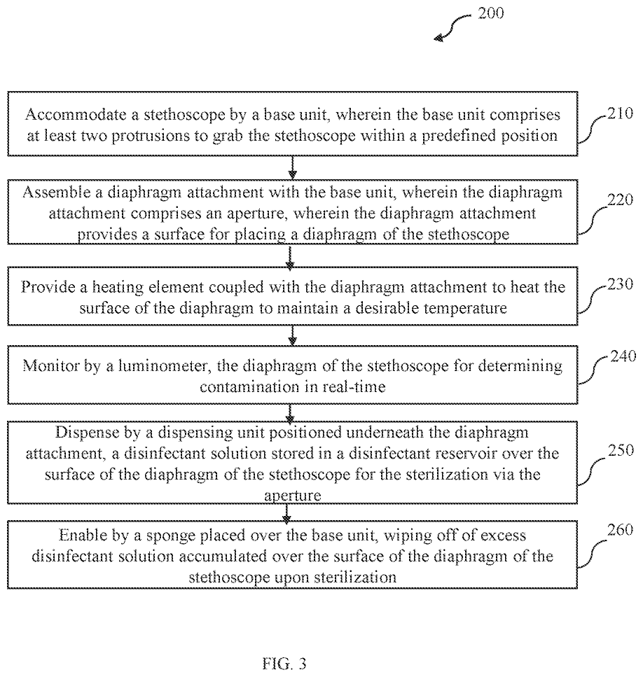

FIG. 3 is a flow chart representing the steps involved in a method 200 for constructing a stethoscope sterilization device in accordance with the embodiment of the present disclosure. The method 200 includes accommodating a stethoscope by a base unit, wherein the base unit comprises at least two protrusions to grab the stethoscope within a predefined position in step 210. In one embodiment, accommodating the stethoscope by the base unit having the at least two protrusions may include accommodating the stethoscope by the base unit having at least two C-shaped protrusions to snap up the stethoscope. In such embodiment, the at least two protrusions grab the stethoscope by enabling pushing and downward movement of the diaphragm of the stethoscope via a spring mechanism.

The method 200 also includes assembling a diaphragm attachment with the base unit, wherein the diaphragm attachment comprises an aperture, wherein the diaphragm attachment provides a surface for placing a diaphragm of the stethoscope in step 220. The method 200 also includes providing a heating element coupled with the diaphragm attachment to heat the surface of the diaphragm to maintain a desirable temperature in step 230. In some embodiment, providing the heating element coupled with the diaphragm attachment to heat the surface of the diaphragm may include providing a coil of heat conducting and transferring heat through a wire wrapped around the diaphragm of the stethoscope.

The method 200 also includes monitoring, by a luminometer, the diaphragm of the stethoscope for determining contamination in real-time in step 240. In one embodiment, monitoring the diaphragm of the stethoscope for determining the contamination may include monitoring the diaphragm of the stethoscope by an adenosine triphosphate (ATP) luminometer.

The method 200 also includes dispensing, by a dispensing unit positioned underneath the diaphragm attachment, a disinfectant solution stored in a disinfectant reservoir over the surface of the diaphragm of the stethoscope for the sterilization via the aperture in step 250. In one embodiment, dispensing the disinfectant solution over the surface of the diaphragm may include dispensing an isopropyl alcohol solution to sterilize the diaphragm of the stethoscope. In such embodiment, the disinfectant solution is collected from the disinfectant reservoir via an inlet pipe. Further the dispensing unit dispenses the disinfectant solution collected over the surface of the diaphragm of the stethoscope via the aperture in a predefined direction to sterilize the diaphragm of the stethoscope. The method 200 also includes enabling, by a sponge placed over the base unit, wiping off of excess disinfectant solution accumulated over the surface of the diaphragm of the stethoscope upon sterilization in step 260.

Various embodiments of the present disclosure provides a medical device which cleans and disinfects a stethoscope before every usage, thereby reduces chances of spread of infection from one patient to another.

Moreover, the present disclosed device utilizes a cheap and simplistic approach for sterilization of the stethoscope by using an easily available disinfecting solution which not only makes the disinfection process easier but also makes it less time consuming. In addition, such a disclosed device is portable and also acts as a stethoscope holder.

Furthermore, the present disclosed device attaches to the stethoscope and the diaphragm sits in the base when on in use. When the stethoscope is being removed for use, the pump or the dispensing unit dispenses a small amount of isopropyl alcohol on to the surface of the diaphragm and excess amount of the isopropyl alcohol is wiped off with the sponge provided. The pump can store enough disinfecting solution for up to 60 uses and can be easily refilled with readily available disinfecting solution.

It will be understood by those skilled in the art that the foregoing general description and the following detailed description are exemplary and explanatory of the disclosure and are not intended to be restrictive thereof.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

1. A stethoscope sterilization device comprising:
a base unit adapted to accommodate a stethoscope, wherein the base unit comprises at least two protrusions to grab the stethoscope within a predefined position;
a diaphragm attachment assembled with the base unit, wherein the diaphragm attachment comprises an aperture, wherein the diaphragm attachment provides a surface for placing a diaphragm of the stethoscope;
a heating element operatively coupled to the diaphragm attachment, wherein the heating element is provided to heat the surface of the diaphragm to maintain a desirable temperature;
a luminometer operatively coupled to the diaphragm attachment, wherein the luminometer is configured to monitor the diaphragm of the stethoscope for determining contamination in real-time;
a dispensing unit positioned underneath the diaphragm attachment, wherein the dispensing unit is configured to dispense a disinfectant solution stored in a disinfectant reservoir over the surface of the diaphragm of the stethoscope for the sterilization via the aperture; and
a sponge placed over the base unit, wherein the sponge enables wiping off of excess disinfectant solution accumulated over the surface of the diaphragm of the stethoscope upon sterilization.

2. The device of claim 1, wherein the at least two protrusions comprises at least two C-shaped protrusions to snap up the stethoscope.

3. The device of claim 1, wherein the at least two protrusions grab the stethoscope by enabling pushing and downward movement of the diaphragm of the stethoscope via a spring mechanism.

4. The device of claim 3, wherein the spring mechanism enables conduction of the diaphragm of the stethoscope from an upward position to downward position thereby triggers the dispensing unit.

5. The device of claim 1, wherein the dispensing unit is adapted to:
compress when the diaphragm of the stethoscope is conducted from the upward position to the downward position;
enable collection of the disinfectant solution from the disinfectant reservoir via an inlet pipe; and
dispense the disinfectant solution collected over the surface of the diaphragm of the stethoscope via the aperture in a predefined direction to sterilize the diaphragm of the stethoscope.

6. The device of claim 1, wherein the heating element comprises a coil of heat conducting and transferring heat through a wire wrapped around the diaphragm of the stethoscope.

7. The device of claim 1, wherein the luminometer comprises an adenosine triphosphate luminometer.

8. The device of claim 1, wherein the disinfectant solution comprises an isopropyl alcohol solution.

9. The device of claim 1, wherein the base unit, the diaphragm attachment and the dispensing unit is fabricated from plastic.

10. A method for constructing a stethoscope sterilization device comprising:
accommodating a stethoscope by a base unit, wherein the base unit comprises at least two protrusions to grab the stethoscope within a predefined position;
assembling a diaphragm attachment with the base unit, wherein the diaphragm attachment comprises an aperture, wherein the diaphragm attachment provides a surface for placing a diaphragm of the stethoscope;
providing a heating element coupled with the diaphragm attachment to heat the surface of the diaphragm to maintain a desirable temperature;
monitoring, by a luminometer, the diaphragm of the stethoscope for determining contamination in real-time;
dispensing, by a dispensing unit positioned underneath the diaphragm attachment, a disinfectant solution stored in a disinfectant reservoir over the surface of the diaphragm of the stethoscope for the sterilization via the aperture; and
enabling, by a sponge placed over the base unit, wiping off of excess disinfectant solution accumulated over the surface of the diaphragm of the stethoscope upon sterilization.
